(19) United States
(12) Patent Application Publication
(10) Pub. No.: US 2006/0243667 A1
(43) Pub. Date: Nov. 2, 2006

(54) Filtrate composition with a radiation absorber

(76) Inventor: Andrew Stone, Boca Raton, FL (US)

Correspondence Address: Peter J. Phillips;Cooper & Dunham LLP, 1185 Avenue of the Americas, New York, NY, 10036, US

(21) Appl. No.: 11/120,014

(22) Filed: May 2, 2005

(51) Int. Cl. A61K 38/17; A61K 31/7048; A61K 31/353; A61K 31/455; A61K 33/00; A61K 31/69; C02F 1/44

(52) U.S. Cl. 210/647; 424/604; 514/2; 514/356; 514/64; 424/722; 514/27; 514/456

(57) ABSTRACT

A filtrate solution composition comprises a vasodilator such as niacin, a high molecular weight protein such as casein, a mineral constituent and a radiation absorber such as boron. The composition may be formed of electrolytes, buffers, a high molecular weight osmotic agent and a radiation absorber. A shock treatment composition may comprise electrolytes, buffers, a rehydrating agent, and a radiation absorber.

### BACKGROUND OF THE INVENTION

The present invention is directed to a composition especially useful for removing toxic matter from the serum of the large intestine, and to a composition for shock treatment, with a radiation absorber.

My prior U.S. Pat. Nos. 5,755,968, 5,620,604 and 6,126,832 relate to methods and systems for dialysis, including filtrate and shock treatment compositions. However, these patents do not discuss use of a radiation absorber in the composition.

### SUMMARY OF THE INVENTION

It is an object of the present invention to replace normal hemodialysis in the filtration of toxic substances in the serum.

It is another object of the invention to provide a filtrate composition with a radiation absorber.

It is a yet further object of the present invention to provide a composition for treating shock.

The invention provides a filtrate composition for use in dialysis comprising a vasodilator, a high molecular weight protein to effect osmotic pressure to achieve diffusion of element across the large intestine membrane into the filtrate, mineral constituents for maintaining proper serum levels in the large intestine, and a radiation absorber.

The dialysis filtrate composition may comprise electrolyte ingredients, buffers, a high molecular weight osmotic agent for removing nitrogenous waste, and a radiation absorber.

The invention also provides a composition for treating shock. The composition for treating shock may comprise electrolyte ingredients, buffers, a rehydrating agent, and a radiation absorber.

### DETAILED DESCRIPTION OF THE PREFERRED EMBODIMENT

According to the invention, a filtrate composition for use in dialysis is provided, comprising a vasodilator a high molecular weight protein to effect osmotic pressure to achieve diffusion of elements across the large intestine membrane into the filtrate, mineral constituents for maintaining proper serum levels in the large intestine, and a radiation absorber.

The vasodilator may be niacin. The high molecular weight protein may have a sufficient molecular weight such that it is not readily absorbed in the colonic mucosa. The high molecular weight protein may be casein. The mineral constituents may be selected from the group comprising sodium chloride, potassium gluconate, magnesium citrate, calcium lactate, ferrous citrate and zinc citrate. The radiation absorber may have a molecular weight large enough so that it will not pass through the colonic mucosa. The radiation absorber may comprise boron.

The radiation absorber may be chelated boron. The radiation absorber may be chelated boron having a molecular weight large enough so that it will not pass through the colonic mucosa.

The composition may further comprise ascorbic acid, lemon bioflavinoids, rutin, hesperidin, acerola, and/or sodium bicarbonate and glucuronic acid to maintain a buffered pH of about 7.38 pH.

The invention also provides a filtrate composition for use in dialysis, comprising electrolytes for establishing electrolyte concentrations, buffers for maintaining acid-base equilibrium, a high molecular weight osmotic agent for removing nitrogenous waste, and a radiation absorber.

The radiation absorber may have a molecular weight large enough so that it will not pass through the colonic mucosa, the radiation absorber may comprise boron. The radiation absorber may be chelated boron. The radiation absorber may be chelated boron having a molecular weight large enough so that it will not pass through the colonic mucosa.

The electrolytes may be selected from the group comprising sodium, potassium, magnesium, calcium and chloride. The buffers may be selected from the group comprising bicarbonate and lactate. The high molecular weight protein may have a sufficient molecular weight such that it is not readily absorbed in the colonic mucosa. The high molecular weight osmotic agent may be selected from the group comprising maltodextrin and casein. The composition according may further comprising a vasodilator.

The composition according may further include an arronium binding ingredient selected from the group comprising activated charcoal and synthetic sorbents.

The composition may further include an ingredient to promote increased creative binding and removal. The ingredient may be zirconium phosphate.

The composition further include an iontophoretic component.

The invention provides a shock treatment solution for use in correcting electrolyte deviation, maintaining acid-base equilibrium and rehydrating the large intestine of a patient, comprising electrolytes for establishing electrolyte concentrations, buffers for maintaining acid-base equilibrium, a rehydrating agent, and a radiation absorber.

The radiation absorber may have a molecular weight large enough so that it will not pass through the colonic mucosa. The radiation absorber may comprise boron. The radiation absorber may be chelated boron. The radiation absorber may be chelated boron having a molecular weight large enough so that it will not pass through the colonic mucosa.

The electrolytes may be selected from the group comprising sodium, potassium, magnesium, calcium and chloride.

The buffers may be selected from the group comprising bicarbonate and lactate.

The rehydrating agent may be selected from the group comprising a saccride and disaccride.

The large intestine is a semi-permeable membrane allowing transport or diffusion or water soluble elements. The purpose of the filtrate solution according to the invention is to provide a vehicle in which undesirable elements or toxins may be removed from the serum of the large intestine without affecting the basic homeostatic mechanisms and important mineral and pH balances. The filtrate composition preferably consists of the following components:

TABLE A

| | | |
|---|---|---|
| Sodium Chloride | 120 | mEq/liter |
| Potassium Gluconate | 5.0 | mEq/liter |
| Magnesium Citrate | 2.4 | mEq/liter |
| Calcium Lactate | 18 | mEq/liter |
| Ferrous Citrate | 220 | mg./liter |
| Zinc Citrate | 205 | mcg./liter |
| Vitamin C (Ascorbic Acid) | 400 | mg./liter |
| Lemon bioflavinoids | 15 | mg./liter |
| Rutin | 15 | mg./liter |
| Hesperidin | 15 | mg./liter |
| Acerola | 15 | mg./liter |
| Niacin | 20 | mg./liter |

Casein (to achieve a filtrate osmolality of 450 mosm/kg) Sodium Bicarbonate (min. of 40 mEq/liter) and Glucoronic Acid to produce a highly buffered pH of about 7.38 pH.
Radiation absorber, such as chelated boron, at about 45 g/deciliter

The mineral constituents serve to maintain proper serum levels of the associated minerals. Niacin is provided for its vasodilator effect and the concomitant effect to increase blood supply to the area, thereby shorting time for serum filtration. Casein is provided to introduce a high molecular weight protein that is not available to transport through the membrane wall, i.e. to effect the osmotic pressure that will achieve diffusion of elements across the membrane into the filtrate. The filtrate is in a water base and is buffered preferably to a pH of about 7.38.

The radiation absorber may be a large boron molecules probably chelated, having a sufficient molecular weight so that it will not pass through or be absorbed by the colonic mucosa. The remaining filtrate may have to be adjusted to compensate for the osmotic pressure of the radiation absorber. It should of course be understood that the concentration values given may be adjusted or changed after clinical testing, to indicate the maximum benefit. The make up of the components may be modified to adjust to individual, metabolic distortions or to sensitivities to the components of the patient.

The present invention provides another embodiment of a dialysis filtrate composition. The objective is to remove about 24 grams of urea daily. The dialysis filtrate composition has as its goals: (1) the re-establishment of proper electrolyte concentrations, (2) maintaining proper acid-base equilibrium, (3) removal of nitrogenous and other associated waste, and (4) absorption of radiation. The dialysis filtrate composition according to this embodiment preferably comprises the following ingredients with the preferred values and ranges indicated:

TABLE B

| | | | | | |
|---|---|---|---|---|---|
| Electrolytes: | Sodium | 135 | mmol/l, range | 134-147 | mmol/l |
| | Potassium | 4 | mmol/l, range | 3-5 | mmol/l |
| | Magnesium | 1 | mmol/l, range | 0.75-2.3 | mmol/l |
| | Calcium | 2 | mmol/l, range | 1-3.5 | mmol/l |
| | Chloride | 105 | mmol/l, range | 95-110 | mmol/l |
| Buffer: | Bicarbonate | 37 | mmol/l, range | 35-45 | mmol/l |
| | Lactate | 8 | mmol/l, range | 0-9 | mmol/l |

High Mol. Weight Osmotic Agent: range 3-16%
Radiation absorber, such as chelated boron, at about 45 g/deciliter

The lactate could be reduced or eliminated, in which case it would preferably be replaced on almost a mmol/l per mmol/l basis by bicarbonate, which could then be increased up to 45 mmol/l if no lactate is used. The bicarbonate is an ideal physiological buffer. The lactate also serves as a buffer, and as a vasodilator.

The high molecular weight osmotic agent can-be any medium weight (e.g., about 200 Daltons) to high molecular weight polymer, protein or amino acid, or combination thereof, that is non-irritating and not readily absorbed in the colonic mucosa. Such examples are maltodextrin (having a molecular weight of 16 k Daltons), and casein.

If necessary or desirable another vasodilator such as niacin in an amount of about 0.25 mg/l may be added to promote increased local/systemic vasodilation.

If necessary or desirable, an ingredient to promote increased ammonium binding may be added to lessen the time necessary for treatment. Such ingredient could be activated charcoal or other synthetic sorbent in an amount of about 15 g/l.

If necessary or desirable, an ingredient may be added to promote increased creatinine binding and removal, such as zirconium phosphate in an amount of about 2 g/l.

To increase the effective removal of cholesterol and triglycerides, the concentration of the osmotic agent can be increased to thereby increase the osmotic pressure, at only a slight increase in risk of irritation, which should be tolerable.

Through the use of the iontophoretic component, antigen/antibody complexes should be removable with or without addition of binding agents.

The present invention also provides a composition for treating shock.

In treating shock the invention has three goals: (1) the correction of any electrolyte composition deviations, (2) the maintenance of proper acid-base equilibrium, (3) rehydration as well as increased serum osmotic pressure to curtail capillary leakage, and (4) absorption of radiation. The shock treatment composition according to the invention preferably comprises the following ingredients:

| | | | | | |
|---|---|---|---|---|---|
| Electrolytes: | Sodium | 135 | mmol/l, range | 134-147 | mmol/l |
| | Potassium | 4 | mmol/l, range | 3-5 | mmol/l |
| | Magnesium | 1 | mmol/l, range | 0.75-2.3 | mmol/l |
| | Calcium | 2 | mmol/l, range | 1-3.5 | mmol/l |
| | Chloride | 105 | mmol/l, range | 95-110 | mmol/l |
| Buffer: | Bicarbonate | 37 | mmol/l, range | 35-45 | mmol/l |
| | Lactate | 8 | mmol/l, range | 0-9 | mmol/l |

Rehydrating Agent: 3-6% by weight
Radiation absorber, such as chelated boron, in the amount of about 45 g/deciliter.

The lactate could be reduced or eliminated, in which case it would preferably be replaced by bicarbonate, which could then be increased almost on a mmol/l per mmol/l basis with the amount of lactate reduced, or up to 45 mmol/l of bicarbonate if no lactate is used.

The rehydrating agent is preferably a non-irritating readily absorbed saccride disaccride, e.g. sorbitol, which would increase the serum osmotic pressure.

While all the above percentages and concentrations described are believed to be appropriate and efficacious, these values may be increased or decreased as the need arises or as may be dictated by clinical trials.

Although one or more preferred embodiments of the system, method and composition according to the present invention have been shown and described, it will be understood that numerous variations and modifications may be effected without departing from the true novel concept and spirit of the present invention. Accordingly, the present invention is not limited to the preferred embodiments disclosed, and is defined by the appended claims.

1. A filtrate composition for use in dialysis, comprising:
a vasodilator;
a high molecular weight protein to effect osmotic pressure to achieve diffusion of elements across the large intestine membrane into the filtrate;
mineral constituents for maintaining proper serum levels in the large intestine; and
a radiation absorber.

2. The composition according to claim 1, wherein the vasodilator is niacin.

3. The composition according to claim 1, wherein the high molecular, weight protein has a sufficient molecular weight such that it is not readily absorbed in the colonic mucosa.

4. The composition according to claim 1, wherein the high molecular weight protein is casein.

5. The composition according to claim 1, wherein the mineral constituents are selected from the group consisting of sodium chloride, potassium gluconate, magnesium citrate, calcium lactate, ferrous citrate and zinc citrate.

6. The composition according to claim 1, wherein the radiation absorber has a molecular weight large enough so that it will not pass through the colonic mucosa.

7. The composition according to claim 1, wherein the radiation absorber comprises boron.

8. The composition according to claim 1, wherein the radiation absorber comprises chelated boron.

9. The composition according to claim 1, wherein the radiation absorber comprises chelated boron having a molecular weight large enough so that it will not pass through the colonic mucosa.

10. The composition according to claim 1, further comprising ascorbic acid.

11. The composition according to claim 1, further comprising lemon bioflavinoids.

12. The composition according to claim 1, further comprising rutin.

13. The composition according to claim 1, further comprising hesperidin.

14. The composition according to claim 1, further comprising acerola.

15. The composition according to claim 1, further comprising sodium bicarbonate and glucuronic acid to maintain a buffered pH of about 7.3 8 pH.

16. A filtrate composition for use in dialysis, comprising:
electrolytes for establishing electrolyte concentrations;
buffers for maintaining acid-base equilibrium;
a high molecular weight osmotic agent suitable for removing nitrogenous waste; and
a radiation absorber.

17. The composition according to claim 16, wherein the radiation absorber has a molecular weight large enough so that it will not pass through the colonic mucosa.

18. The composition according to claim 16, whereinin the radiation absorber comprises boron.

19. The composition according to claim 16, wherein the radiation absorber comprises chelated boron.

20. The composition according to claim 16, wherein the radiation absorber comprises chelated boron having a molecular weight large enough so that it will not pass through the colonic mucosa.

21. The composition according to claim 16, wherein the electrolytes are selected from the group consisting of sodium, potassium, magnesium, calcium and chloride.

22. The composition according to claim 16, wherein the buffers are selected from the group consisting of bicarbonate and lactate.

23. The composition according to claim 16, wherein the high molecular weight protein has a sufficient molecular weight such that it is not readily absorbed in the colonic mucosa.

24. The composition according to claim 16, wherein the high molecular weight osmotic agent is selected from the group consisting of maltodextrin and casein.

25. The composition according to claim 16, further comprising a vasodilator.

26. The composition according to claim 16, further including an ammonium binding ingredient selected from the group consisting of activated charcoal and synthetic sorbents.

27. The composition according to claim 16, further including an ingredient to promote increased creatinine binding and removal.

28. The composition according to claim 27, wherein the ingredient is zirconium phosphate.

29. The composition according to claim 15, further including an iontophoretic component.

30. A shock treatment solution for use in correcting electrolyte deviation, maintaining acid-base equilibrium and rehydrating the large intestine of a patient, comprising:
electrolytes for establishing electrolyte concentrations;
buffers for maintaining acid-base equilibrium;
a rehydrating agent; and a radiation absorber.

31. The solution according to claim 30, wherein the radiation absorber has a molecular weight large enough so that it will not pass through the colonic mucosa.

32. The solution according to claim 30, wherein the radiation absorber comprises boron.

33. The solution according to claim 30, wherein the radiation absorber comprises chelated boron.

34. The solution according to claim 30, wherein the radiation absorber comprises chelated boron having a molecular weight large enough so that it will not pass through the colonic mucosa.

35. The solution according to claim 30, wherein the electrolytes are selected from the group consisting of sodium, potassium, magnesium, calcium and chloride.

36. The solution according to claim 30, wherein the buffers are selected from the group consisting of bicarbonate and lactate.

37. The solution according to claim 30, wherein the rehydrating agent is selected from the group consisting of a saccharide and disaccharide.

38. A method of performing dialysis using a filtrate composition comprising a radiation absorber.

39. The method according to claim 38, wherein the radiation absorber comprises boron.

40. The method according to claim 38, wherein the radiation absorber comprises chelated boron.

41. The method according to claim 38, wherein the radiation absorber comprises chelated boron having a molecular weight large enough so that it will not pass through the colonic mucosa.
